(19) United States
(12) Patent Application Publication
(10) Pub. No.: US 2014/0276086 A1
(43) Pub. Date: Sep. 18, 2014

(54) DEFLECTABLE IVUS CATHETER

(71) Applicant: VOLCANO CORPORATION, San Diego, CA (US)

(72) Inventor: David G. Miller, North Andover, MA (US)

(73) Assignee: VOLCANO CORPORATION, San Diego, CA (US)

(21) Appl. No.: 14/208,184

(22) Filed: Mar. 13, 2014

Related U.S. Application Data
(60) Provisional application No. 61/782,407, filed on Mar. 14, 2013.

(51) Int. Cl. A61B 8/00

(52) U.S. Cl. CPC A61B 8/445
USPC 600/467

(57) ABSTRACT

The invention generally relates to intravascular intervention catheters and provides catheters for performing an intravascular procedure that use a mechanism to control an operation at the end of the catheter. The mechanism can be used to steer the distal tip of the catheter from a controller at a proximal end of the catheter. The mechanism may include one or more pull wires extending along the catheter. The mechanism may optionally be manually or computer controlled.

### CROSS-REFERENCE TO RELATED APPLICATION

This application claims the benefit of, and priority to, U.S. Provisional Patent Application No. 61/782,407, filed Mar. 14, 2013, the contents of which are incorporated by reference.

### FIELD OF THE INVENTION

The invention generally relates to intravascular intervention catheters and systems and methods for guiding catheter operations.

### BACKGROUND

Some people are at risk of having a heart attack or stroke due to fatty plaque buildups in their arteries that restrict the flow of blood or even break off and block the flow of blood completely. A number of intravascular procedure have the potential to treat those plaque buildups. For example, angioplasty involves delivering a balloon or a stent to open up the constricted blood vessel. This procedure involves inserting a thin catheter into the patient's vessel and navigating it to the affected site.

Where the plaque has built up to the point that it effectively closes off the vessel, the affected sit is sometimes referred to as a chronic total occlusion. Such an occlusion can be treated by a catheter with a mechanism at the tip that is designed to ablate the plaque, opening a hole through the occlusion, allowing the person's blood to flow and carry oxygen and nutrients throughout the body.

Using a catheter in an intravascular intervention to treat plaque buildup carries some difficulties. The blood vessels define a convoluted and intricate network. Simply navigating a catheter to the correct site from outside the body can be very difficult.

### SUMMARY

The invention provides catheters for performing an intravascular procedure. The invention provides a mechanism to control a distal tip of the catheter from a controller at a proximal end. In particular, the deflection mechanism may be employed on a phased-array (e.g., non-rotating) catheter, such as an IVUS catheter. A non-rotation catheter is preferable, since the simplicity of its construction allows the deflection mechanism to be included with ease and low cost. Using a catheter of the invention, a doctor has precision control over steering and operations. Thus catheter of the invention can be used in an intravascular treatment with greater ease and reliability. Thus, a greater number of people can be treated for arterial plaque, and life-threatening heart attacks and strokes can be prevented.

In certain aspects, the invention provides a system for guiding an intravascular operation that uses a tip deflection mechanism at a distal portion of a catheter that is controlled from a proximal portion. The catheter may include one or more pull wires that may extend from a distal portion of the catheter to a proximal portion. At the proximal portion, the pull wires may terminate in a controller such as a joystick, or in one or more servomotors (e.g., under the control of a computer system). Applying tension to a pull wire at the proximal end may tend to deflect the catheter to tip towards the side where the distal end of the pull wire terminates. In a preferred embodiment, the catheter is an over-the-wire style, non-rotating imaging catheter.

The catheter may include a flexible tube inner member to provide one or more guidewire lumen.

In some embodiments, the catheter is steered under computer control. The catheter takes an image; the computer detects a blood-plaque border and steers the catheter towards the border; the catheter takes an image, and the system moves in to the feature of interest. In certain embodiments, feedback from the computer system is used to shut down an operation or guide a distal tip of a catheter away from a feature—e.g., for patient safety.

In certain aspects, the invention provides a system for performing an intravascular procedure. The system includes a catheter for insertion into a blood vessel, an imaging device at a distal portion of the catheter, and a controllable deflection mechanism at a distal tip of the catheter. The system includes a control device to receive the steer the catheter an in intravascular operation. The catheter may be steered away from a wall of the vessel, steered a branch of a bifurcated vessel, used for the ablation of plaque, any other operation, or a combination thereof.

A computer may be included to detect a feature in the vessel and provide a signal to guide the catheter. The computer may be configured to automatically detect a border within the vessel. In some embodiments, the system includes an ablation mechanism, such as a cutter tip or an RF transmitter. The computer and control device can work to prevent any ablation operations within a determined distance of the detected border.

In related aspects, the invention provides a method for performing an intravascular procedure that includes inserting a catheter for insertion into a blood vessel, taking a picture of the vessel with an imaging device on the catheter, and steering the catheter with a control mechanism. Preferably, the picture is taken via IVUS and the catheter is a non-rotating catheter.

### BRIEF DESCRIPTION OF THE DRAWINGS

FIG. 1 illustrates an imaging system according to certain embodiments.

FIG. 2 shows a portion of the catheter.

FIG. 3 diagrams an embodiment of the invention.

FIG. 4 illustrates coupled interactions between a user and a computing device.

FIG. 5 illustrates a display of an imaging system showing a luminal border.

FIG. 6 depicts a defined area around point on a tomographic view.

FIG. 7 shows a corresponding B-scan.

### DETAILED DESCRIPTION

The present invention provides a system and method of using an intravascular imaging system that includes controlling operations such as steering or ablation performed with a catheter. Systems and methods of the invention operate with intravascular imaging systems such as, for example, intravascular ultrasound (IVUS) system, preferably a non-rotating phased array system.

FIG. 1 illustrates an exemplary imaging system 101 in accordance with one embodiment of the present invention. System 101 is described for illustrative purposes as an IVUS system. It will be appreciated that detection methods described herein can operate with a 3D data set collected via other imaging modalities as well. System 101 includes console 110 electrically connected to a computing device 120 and a distal tip 114 via a catheter 112 and patient interface module 105. The distal tip 114 is inserted into a blood vessel of a patient lying etherized upon a table and used to gather image data (i.e., blood-vessel data, or data that can be used to identify the shape of a blood vessel, its density, its composition, etc.). The image data are then provided to (or acquired by) the computer device 120, where they are used to produce an image of the vessel. Systems for IVUS suitable for use with the invention are discussed in U.S. Pat. No. 5,771,895; U.S. Pub. 2009/0284332; U.S. Pub. 2009/0195514; U.S. Pub. 2007/0232933; and U.S. Pub. 2005/0249391, the contents of each of which are hereby incorporated by reference in their entirety.

Catheter 112 is a steerable catheter and optionally includes a steering mechanism and PIM 105 includes a control device operable to steer catheter 112. Catheter 112 may include any suitable steering mechanism. In some embodiments, a steering mechanism includes a plurality (e.g., 4) of extended pull wires 225 beneath a surface of catheter 112, extending along the catheter body and disposed around it. Pulling back on any one of the pull wires 225 tends to cause the tip 114 steer in a direction of the pulled-back wire. The wire may include a metal, such as stainless steel or nitinol, or any other material including, for example, a polymer, such as polytetrafluoroethylene or a plastic. A proximal end of each wire can be wound onto a spindle mounted on an axle of a servomotor. Each motor may be wired to a chip coupled to computer device 120. A program in computer device 120 can drive each motor according to a pattern that causes the motors to wind up the reins, thereby pulling them back, and steering catheter 112. In certain embodiments, a steering mechanism includes the use of an electroactive polymer disposed within catheter 112 (e.g., substantially as just described for reins). Contraction of the polymers is controlled by computer 120 being used to cause the application of a potential difference across individual ones of the electroactive polymers. By referring to information, commands, or both, in a program, computer 120 can steer catheter 112 via the electroactive polymer.

FIG. 2 shows a catheter 112 of the invention, with the flexible protective cover removed. Catheter 112 includes at least one pull wire 225 extending towards tip 114. A phased array of transducers 209 is disposed near tip 114. A coupling 213 connects the pull wire 225 to the catheter. Each pull wire 225 extends under catheter body 217 (e.g., on the outside of guidewire lumen 221).

Preferably, catheter 112 includes a phased array IVUS transducer and associated electronics (e.g., one or more PMUT “tadpoles”). Pull wire 225 may be attached to a lever arm at the tip of the catheter. Pull wire 225 may extend to the proximal controller and terminate at an attachment to handle, which can be actuated by a user.

In FIG. 2, the distal end of catheter 112 is shown with a conical tip around the guidewire lumen. The lumen runs through the center of catheter. Next is the transducer and electronics and next is the coupling to which pull wire 225 is attached. Next is the coupling which functions as a hinge to permit the tip to deflect in response to tension and displacement of the pull wire. Next the coupling is joined to the catheter body. The inner member may be a flexible tube which provides the central lumen in the catheter for the guidewire. The flexible sheath that covers the coupling and the electrical cable wires are not shown.

In some embodiments, a distal tip 114 includes a device such as an IVUS imaging device, an ablation mechanism, steering mechanics, or a combination thereof. Where, for example, an imaging device is included, catheter 112 can be used to capture an image of a patient's vessel.

In certain embodiments, an ablation mechanism is included to open a hole through a chronic total occlusion. In some embodiments, the invention provides systems and methods for treating a chronic total occlusion. Catheter 112 can be used to bring an imaging device (e.g., such as a forward looking IVUS device) to a chronic total occlusion. Once the tip 114 is positioned appropriately, an ablation mechanism is activated. Any suitable mechanism for ablating plaque may be used. For example, RF waves can be used to ablate and break through a chronic total occlusion. Other suitable ablation mechanisms known in the art include mechanical ablation mechanisms (e.g., drills, cutter, knives, Archimedes' screws, punches, slicers, etc.), lasers, or others. In certain embodiments, computer 120 includes a processor and memory for analyzing signal captured by an imaging device. For example, computer 120 may do a virtual histology analysis on an image and determine a location of a lumen, necrotic tissue, healthy tissue, and other features within a patient's vessel. By determining where an outer border of a vessel is, where a lumen is, and other information, a computer can then determine (e.g., mathematically), where a center of vessel should be, and thus, where a center of a lumen should be. Additionally or alternatively, a computer can determine where a portion of catheter 112 (e.g., an imaging device or an ablation mechanism) presently is. Computer 120 can use this information to guide the operation of catheter 112. For example, if an ablation mechanism is within a determined distance (e.g., 0.5 mm) of a should-be center of a vessel, and is also within necrotic tissue or plaque, computer 120 can determine to operate the ablation mechanism. Computer 120 can send a signal that is received by a control device (e.g., within PIM 105) that then operates the mechanism on catheter 112. Additionally or alternatively, computer 120 can issue instructions to steer catheter 112 towards an occlusion, towards a center of a vessel, or in other desired ways (e.g., down a desired branch at a vessel bifurcation).

In certain embodiments, processor and memory systems in computer 120 are operable to receive an image, determine an outer border, determine a blockage, determine a putative healthy lumen, determine a center of a lumen, and calculate what should be a center. Using computer 120 to figure out a delta between center of lumen and what should be a center of a lumen, systems and methods of the operation can move a tool to the should-be center, or to the edge of a plaque or occlusion, and ablate the material away. In some applications, during a burst of RF energy, an imaging component will not produce a viewable image due to the interference of the RF energy. One function that can be provided by computer 120 is to alternate between imaging and blasting. This way, energy from RF blasting does not interfere with imaging operations.

Additionally, systems and methods of the invention provide important safety functionality. For example, computer 120 can issue signals that prevent any ablation operation if a device is with a certain distance from a vessel wall. As will be appreciated from the disclosure herein, the invention provides systems and methods to coordinate treatment operations as well as imaging operations, particularly through the use of computer-guided steering of an intravascular catheter.

Where IVUS is the imaging modality, IVUS data are gathered in segments, either through a rotating transducer or an array of circumferentially positioned transducers, where each segment represents an angular portion of an IVUS image. Thus, it takes a plurality of segments (or a set of IVUS data) to image an entire cross-section of a vascular object. Furthermore, multiple sets of IVUS data are typically gathered from multiple locations within a vascular object (e.g., by moving the transducer linearly through the vessel). These multiple sets of data can then be used to create a plurality of two-dimensional (2D) images or one three-dimensional (3D) image. It should be appreciated that the present invention is not limited to the use of an IVUS device (or the acquisition of IVUS data), and may further include using thermographic devices, optical devices (e.g., an optical coherence tomography (OCT) console), MRI devices, or any vascular imaging devices generally known to those skilled in the art. For example, instant automatic border detection may be provided in OCT systems such as those described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety. It should further be appreciated that the computing device depicted in FIG. 1 includes, but its not limited to, personal computers or any other data-processing devices (general purpose or application specific) that are generally known to those skilled in the art.

FIG. 3 shows an embodiment using a system of the invention for crossing a chronic total occlusion. Catheter 112 is navigated to a target site within the vessel (e.g., by a doctor using angiographic guidance, by a computer, by the guidance offered by the imaging modality, or a combination thereof). Once the navigation is received, the system acquires an image of the vessel. Additionally, the system receives information about an intended operation (e.g., is this an ablation operation, a steering procedure, etc.). The image is then analyzed. The image data (or multiple sets thereof) are provided to (or acquired by) the computing device 120. A portion of the 3D data set may be displayed for the user on, for example, monitor 103. The display will show, in a cross section of a blood vessel, objects within a certain range of transducer 114.

One or more features are detected by computer 120 within the vessel. Vascular objects that can be detected includes vessel walls and occlusions. Having detected the occlusion, computer 120 determines an ablation target within the occlusion and navigates an ablation mechanism at tip 114 of catheter 112 to the target. Once the ablation mechanism is in place, it is activated and the occlusion is ablated.

FIG. 4 illustrates the use of systems and methods of the invention to provide a safety operation in a procedure with an objective of crossing a chronic total occlusion.

An operator/user, such as a physician, views the display 103 to see images from a 3D data set. The user uses joystick 125 to navigate to an occlusion. The user then operates a trigger on the outside of the system to run the ablation mechanism.

Computing device 120 detects the occlusion as well as the wall. Based on determined parameters stored in memory, computing device 120 defines a dead zone for patient safety (e.g., no laser ablation mechanism should be operated within 0.1 mm of a vessel wall). Computing device 120 detects that the operator seeks to operate the ablation mechanism within the dead zone and overrides the ablation command. Computer 120 may further, optionally, steer catheter 112 to nudge the tip out of the dead zone. The operator may view the vessel and see that operations as determined by computer 120 have been implemented. The operator may then proceed to perform the occlusion-crossing procedure safely.

FIG. 5 illustrates, in simplified fashion, a display 131 of an imaging system showing a luminal border 320 and a medial border 310. In certain embodiments, the system uses a processor to perform an image processing operation to detect a border. A border may be detected instantly, automatically, solely in response to navigational input or cessation of navigational input, or a combination thereof. Automatically generally refers to an absence of human intervention. Where a system automatically provides a border in response to navigational input, that means that no human action other than the navigational input is required. Instant can mean simultaneously, substantially simultaneously, within a few microseconds, within about a second, or within a few seconds. Any suitable border detection algorithm can be employed. Exemplary border detection systems are discussed in U.S. Pat. No. 7,463,759; U.S. Pat. No. 6,475,149; U.S. Pat. No. 6,120,445; U.S. Pub. 2012/0226153; and U.S. Pub. 2007/0201736, the contents of which are incorporated by reference. For example, in some embodiments, the system uses a radius to detect a control point; uses the control point to define a search area; uses the search area to find a portion of a border; and uses the portion of the border to locate an entire border. Looking at FIG. 5, a first control point 22 may be taken as a point of highest contrast on an arbitrary radius 137 from the center of the screen to an edge (e.g., the “due east” radius at a theta of zero). Starting from the control point 22, system then defines an area 25 around point 22.

FIG. 6 depicts a defined area 25 around point 22. Area 25 operates as a search window. The search window area 25 may be a rectangle, circle, ellipse, polygon, or other shape. It may have a predetermined area (e.g., a certain number of pixels). In some embodiments, a size and shape of area 25 is determined by a combination of input device resolution, screen area subtended by a pixel at the particular polar coordinates, current zoom factor, usability studies, or a combination thereof. Usability studies can be performed to establish a statistical model of user repeatability and reproducibility under controlled conditions. Computer 120 may determine that all areas within, for example, 0.2 mm of border 310 are a dead zone and prevent ablation there.

FIG. 7depicts a defined area 25 around point 22 shown in a B scan. The system searches for the border within area 25 by performing a processing operation on the corresponding data. The processing operation can be any suitable search algorithm known in the art. In some embodiments, a morphological image processing operation is used. Morphological image processing includes operations such as erosion, dilation, opening, and closing, as well as combination thereof. In some embodiments, these operations involve converting the image data to binary data giving each pixel a binary value. With pixels within area 25 converted to binary, each pixel of a feature such as a border may be black, and the background pixels will predominantly be white (or vice versa). In erosion, every pixel that is touching background is changed into a background pixel. In dilation, every background pixel that is adjacent to the non-background object pixels is changed into an object pixel. Opening is an erosion followed by a dilation, and closing is a dilation followed by an erosion. Morphological image processing is discussed in Smith, The Scientist and Engineer's Guide to Digital Signal Processing, 1997, California Technical Publishing, San Diego, Calif., pp. 436-442.

If a border is not found within area 25, area 25 can be increased and the increased area can be searched. This strategy can exploit the statistical properties of signal-to-noise ratio (SNR) by which the ability to detect an object is proportional to the square root of its area. See Smith, Ibid., pp. 432-436.

With reference to FIG. 7, once a portion of the border is detected within area 25, the search can then be extended “upwards” and “downwards” into adjacent A-scan lines in the B-scan until the entire border is detected by the processor and its location is determined with precision. In some embodiments, image processing operations incorporate algorithms with pre-set parameters, user-set parameters, or both that optimize results and continuity of results. For example, if a line appears that is not contiguous across an entire 100% of the image (e.g., the entire extent of the B-scan or a full circle in a tomographic view), an accept or reject parameter can be established based on a percent contiguous factor. In some embodiments, lines that are contiguous across less than 75% (or 50% or 90%, depending on applications) are rejected while others are accepted.

While certain methods of using a processor to detect a feature are described above, described steps can be performed in other orders. For example, the system can apply morphological processing operations to an entire image and detect every element, or every element that satisfies a certain quality criterion. Then the system can receive use input (e.g., navigation) and respond by provided the pre-detected border. Similarly, the steps can be performed simultaneously. Using the methodologies herein, systems of the invention can provide a border detected within an image of an imaging system, such as an IVUS system, with great precision, based on a location that an operator navigates too. As discussed above, any suitable border detection process can be employed. Border detection is described, for example, in U.S. Pat. No. 8,050,478; U.S. Pat. No. 7,068,852; U.S. Pat. No. 6,491,636; U.S. Pub. 2011/0216378; and U.S. Pub. 2003/0016604, the contents of which are incorporated by reference.

Mechanisms and principles for steering and guiding catheters are discussed in U.S. Pat. No. 7,666,204; U.S. Pat. No. 7,048,711; U.S. Pat. No. 5,916,194; U.S. Pat. No. 5,803,083; U.S. Pat. No. 5,358,478; U.S. Pub. 2012/0253276; U.S. Pub. 2006/0270976; U.S. Pub. 2004/0260236;

As used herein, the word “or” means “and or or”, sometimes seen or referred to as “and/or”, unless indicated otherwise.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A steerable catheter comprising:
an extended body comprising a proximal portion and a distal portion;
a lumen throughout the length of the extended body;
at least one pull wire extending from the proximal portion to the distal portion; and
a controller mechanism disposed at the proximal portion.

2. The catheter of claim 1, further comprising a phased array of ultrasonic transducers.

3. The catheter of claim 1, wherein the catheter is an over-the-guidewire style IVUS imaging catheter.

4. The catheter of claim 1, wherein the catheter is a non-rotating, over-the-guidewire style IVUS imaging catheter.

5. The catheter of claim 1, wherein the controller mechanism comprises a joystick.

6. The catheter of claim 1, wherein the controller mechanism comprises a computer comprising a tangible, non-transitory memory coupled to a processor.

7. The catheter of claim 6, wherein the computer is configured to automatically detect an intravascular feature.
